(19) United States
(12) Patent Application Publication
(10) Pub. No.: US 2024/0101644 A1
(43) Pub. Date: Mar. 28, 2024

(54) THE USE OF INTRAVENOUS IMMUNOGLOBULIN IN THE PREPARATION OF A DRUG FOR THE TREATMENT OF CUTIS VERTICIS GYRATA DISEASE

(71) Applicants: BURSA ULUDAG ÜNIVERSITESI, Nilüfer, Bursa (TR); NOBEL ILAC SANAYI VE TICARET A.S., Ümraniye, Istanbul (TR)

(72) Inventor: Serkan YAZICI, Nilüfer, Bursa (TR)

(21) Appl. No.: 18/263,161

(22) PCT Filed: Dec. 28, 2020
(86) PCT No.: PCT/TR2020/051411

§ 371 Date: Jul. 27, 2023

(30) Foreign Application Priority Data
Nov. 12, 2020 (TR) 2020/18118

(51) Int. Cl. C07K 16/06; A61P 17/00

(52) U.S. Cl. CPC C07K 16/06; A61P 17/00; A61K 2039/507

(57) ABSTRACT

Disclosed is the use of intravenous immunoglobulin in the preparation of a drug for the treatment of Cutis Verticis Gyrata disease.

### TECHNICAL FIELD

The invention relates to a perfusion cannula with vascular grafts.

In particular, the invention relates to the use of intravenous immunoglobulin (IVIG) in the preparation of a medicament for treating Cutis Verticis Gyrata (CVG) disease.

### PRIOR ART

Cutis Verticis Gyrata (CVG), also called Paquidermia verticis gyrata, cutis verticis plicata, and “bulldog” scalp syndrome, is a medical condition usually associated with the excessive soft-tissue proliferation or thickening of the scalp. In this disease the SCALP is similar in appearance to cerebral cortex gyri, is characterized by the formation of ridges on the scalp, and is a rare skin disease. This disease, which is more common in men, usually occurs after puberty but before the age of 30. The number of folds on SCALP can vary from roughly two to ten and are typically soft and spongy. These ridges cannot be flatten with pressure. The condition typically affects the central and posterior regions of the SCALP, although there are occasionally cases where it affects the entire SCALP.

Although its etiopathogenesis is not fully known, its primary forms associated with neuropsychiatric or ophthalmological abnormalities and its secondary forms associated with some localized or systemic inflammatory or neoplastic diseases have been described. Histopathologically, primary CVG is characterized by an increase in the number and size of collagen bundles and an increase in the dermal matrix.

CVG treatment depends on the etiology, the extent of SCALP involvement, and the preference of the patient. While the treatment of the underlying disorder in secondary CVG generally causes CVG to regress, local SCALP hygiene in primary CVG prevents secretion accumulation, maceration, unpleasant odor, and is also important to prevent secondary infections. Although primary CVG is a benign condition, surgical treatment is applied especially in advanced cases due to psychological and cosmetic concerns, however it is not curative and repeated surgeries are required. In the current technique, there are studies for CVG treatment.

One of these studies is the United States patent application numbered US20180311499A1. This application relates to the treatment of dermatological conditions, including CVG disease, by neuromodulation. The invention disclosed herein includes administering to a subject an effective amount of a neuromodulatory agent to treat the subject for the dermatological condition. Also compositions, kits and systems for the implementation of these methods are described. The term “neuromodulatory agent” is used broadly to refer to any agent, which agent may be a pharmacological or electrical agent (i.e., electrical stimulus), to influence or change nervous system activity, e.g., in a manner effective to treat a target dermatological condition. Accordingly, the agents that can be used in the method of the invention are: neuromodulatory agents, pharmacological agents with neuromodulatory activity. Pharmacological agents having neuromodulatory activity that may be employed in methods of the invention include, but are not limited to: cholinergic system modulators, serotonin system modulators, dopamine system modulators, noradrenaline system modulators, histamine system modulators, neuropeptide system modulators, substance P system modulators, adenosine system modulators, GABA system modulators, opioid peptides system modulators, oxytocin system modulators, etc.

Patent application number WO2009093119 relates to the use of serine protease inhibitors in the treatment of skin diseases, including CVG. The present invention describes therapeutic compounds that are inhibitors of serine proteases, their pharmaceutical compositions, and their use in the treatment of the human or animal body. More specifically, the present invention relates to a method for the treatment, diagnosis or prognosis of skin diseases comprising the administration to a subject in need thereof of a therapeutically effective amount of a Serine protease inhibitor. Here, the Serine protease inhibitor is a recombinant Serine protease inhibitor, is selected from the group consisting of SEQ ID Nos. 2, 4, 6, 8, 10, 12, and 14 or a biologically active fragment thereof with a Serine protease inhibitory activity.

Although there are studies on the treatment of this disease in the prior art, an effective medical treatment of CVG is not reported in the current literature.

As a result, due to the above-mentioned drawbacks and the inadequacy of the existing solutions, an improvement in the technical field has been required.

### THE PURPOSE OF INVENTION

The present invention relates to the use of intravenous immunoglobulin that meets the above-mentioned requirements, eliminates all disadvantages, and brings some additional advantages.

The main purpose of the invention is to obtain significant clinical and radiological responses against primary CVG disease, especially a reduction in SCALP thickness, by the use of IVIG.

Another purpose of the invention is to achieve effective and safe results in CVG disease with the use of IVIG.

In order to fulfill the purposes described above, the invention relates to the use of IVIG in the preparation of a medicament for treating CVG disease.

The structural and characteristic features of the invention and all advantages thereof will be more clearly understood by means of the following detailed description. Therefore, the evaluation should be made considering this detailed description.

### DETAILED DESCRIPTION OF THE INVENTION

In this detailed description, the use and the preferred embodiments of the IVIG of the invention are explained only for a better understanding of the subject matter and without any restrictive effect.

The invention is the use of IVIG in the preparation of a drug for the treatment of CVG disease. Besides, IVIG has been developed for use in the treatment of CVG disease.

After six cures of treatment with the administration of the invented medicament, significant regression in the ridged appearance, reduction in the depth of the ridges, the superficiality of the lesions, and hardening of the skin consistency were observed clinically. In addition, radiological examinations regarding the SCALP thickness after six cures of treatment were performed by the same experienced radiologist. Sections were taken from the midsagittal region in the T1 image, and a decrease in the number of SCALP folds was observed in the occipital region, and a decrease in vertical measurements of approximately the same levels between SCALP-external takula was detected. The results obtained on 20 Apr. 2019 (before application) and 28 Oct. 2020 (after application) are shown in Table-1.

TABLE 1

| Cranial | Coronal | | Parietal | Parietal | Occipital |
|---|---|---|---|---|---|
| MRI | Suture | Vertex | Bone | Bone | Protuberance |
| 20 Apr. 2019 | 1.44 | 1.87 | 1.65 | 1.30 | 0.90 |
| 28 Oct. 2020 | 1.21 | 1.83 | 1.53 | 1.20 | 0.61 |

In addition, a formulation for the treatment of CVG disease is being developed with the invention, and the formulation contains IVIG in its most basic form. In the invention, IVIG is used in a formulation for treating CVG disease. In a preferred embodiment of the invention, the said formulation is administered intravenously.

In a preferred embodiment of the invention, IVIG or the formulation containing it for the treatment of CVG disease is administered at a total dose of 2 g/kg in a month. This amount is divided equally into five days and applied in daily doses of 0.4 g/kg. Preferably, 0.5 ml/kg/hour infusion is started and as the patient tolerates, it is administered as a slow intravenous infusion for 6-8 hours, increasing to a maximum of 4 ml/kg/hour every 30 minutes. Apart from the general infusion rules, there is no additional procedure that should be applied.

The use of IVIG disclosed by the invention is used effectively and safely in the treatment of hypogammaglobulinemia, antibody deficiency disorders, other immunodeficiency conditions, and some skin diseases due to its several immunosuppressive and anti-inflammatory properties that include modulation of immunoglobulin G (IgG) levels, lymphocyte, and reticuloendothelial functioning, cytokine production, complement regulation and clearance of pathogenic IgG. As a result of the tests and examinations performed, it is seen that a significant clinical and radiological response (reduction in SCALP thickness) was obtained in the primary CVG case using IVIG. The use of IVIG has been found to be effective and safe in CVG disease.

1. Use of intravenous immunoglobulin in the preparation of a medicament for the treatment of Cutis Verticis Gyrata disease.

2. Intravenous immunoglobulin for the treatment of Cutis Verticis Gyrata disease.

3. Use of intravenous immunoglobulin in a formulation for treating Cutis Verticis Gyrata disease.

4. A formulation for the treatment of Cutis Verticis Gyrata disease comprising intravenous immunoglobulin.

5. The formulation according to claim 4, wherein it is administered intravenously.

6. A method of use according to claim 1, wherein it is administered at a total dosage of 2 g/kg per month.

7. The formulation according to claim 4, wherein it is administered at a total dosage of 2 g/kg per month.

8. A method of use according to claim 3, wherein it is administered at a total dosage of 2 g/kg per month.
